(19) United States
(12) Patent Application Publication
(10) Pub. No.: US 2007/0267026 A1
(43) Pub. Date: Nov. 22, 2007

(54) PROTECTIVE COVERING FOR MEDICAL INSTRUMENTS

(76) Inventor: Grace A. Grant-Jennings, Austin, TX (US)

Correspondence Address: ZAGORIN O'BRIEN GRAHAM LLP, 7600B NORTH CAPITAL OF TEXAS HIGHWAY, SUITE 350, AUSTIN, TX, 78731, US

(21) Appl. No.: 11/419,357

(22) Filed: May 19, 2006

(51) Int. Cl. A61F 5/37; A61B 19/08

(52) U.S. Cl. 128/846; 128/852

(57) ABSTRACT

Although protective coverings for medical instruments are known in the art, they are difficult or even impossible for the user to remove without risking contamination of the instrument or the user. An improved protective covering provides for an unassisted user to remove it with a single gloved, even contaminated, hand. In some embodiments, the invention provides a disposable protective covering for reusable medical instruments and devices that can be easily and quickly applied during donning of personal protective equipment (PPE), and easily and quickly removed during removal of PPE, without risking inadvertent contamination of the instrument or device. In some embodiments, a disposable protective covering includes a fastening means that secures a sheath to the instrument during instrument use, and that is releasable in a single motion by a moderate force applied toward the distal end of the covering from a point along the distal portion of the covering.

### BACKGROUND

1. Field of the Invention

This invention relates to protective coverings for medical instruments.

2. Description of the Related Art

Health care environments play host to a multitude of infectious agents, in addition to health care personnel and patients. These pathogens can be transmitted by direct, indirect, and droplet contact. In addition to contact transmission, pathogenic microorganisms can be transmitted through contaminated air or by a common vehicle. Direct contact refers to body surface-to-body surface contact and physical transfer of microorganisms between a susceptible host and an infected, colonized, or contaminated person. Indirect contact refers to contact with a contaminated object, e.g., instruments, hands. Transmission by droplet contact occurs when conjunctival, nasal, or oral mucosa come into contact with droplets containing microorganisms generated from an infected person (by coughing, sneezing, and talking) that are propelled a short distance (influenza). In contrast, airborne transmission refers to contact with droplet nuclei containing microorganisms that can remain suspended in the air for long periods or to contact with dust particles containing an infectious agent (chicken pox) that can be widely disseminated by air currents. Contact with contaminated items such as food, water, medications, devices, and equipment may result in common vehicle transmission. (See E. A. Bolyard, et al., “Guideline for Infection Control in Health Care Personnel, 1998,” AJIC: American Journal of Infection Control, vol. 26, no. 3, June 1998, p. 292.)

Increased use of antibiotics has resulted in the emergence of resistant organisms. The chief risk factor for colitis caused by Clostridium difficile is, in fact, antibiotic treatment. Infections by methicillin resistant Staphylococcus aureus (MRSA) and vancomycin resistant Enterococcus species (VRE) are becoming more widespread and more frequently encountered. Diseases caused by these agents may be essentially untreatable, so prevention is the best, and sometimes only, defense.

To protect patients and health care personnel from nosocomial infections, standards for use of personal protective equipment (PPE) have been developed. For example, infections that can be transmitted by contact with blood and other potentially infectious materials (OPIM) include: human immunodeficiency virus (HIV); Hepatitis A (HAV), B (HBV), and C (HCV); Staphylococcus and Streptococcus infections; gastroenteritis due to Salmonella and Shigella species; pneumonia; syphilis; tuberculosis (TB); malaria; measles; chicken pox; herpes; urinary tract infections; and blood infections.

Guidelines developed by the Occupational Safety and Health Administration of the U.S. Department of Labor (OSHA), known as “universal procedures,” call for health care personnel “[to treat] all human blood and certain body fluids . . . as if known to be infectious” (29 CFR 1910.1030(b)). Health care personnel must don protective gowns, masks, and latex or vinyl gloves when exposure to blood or OPIM is expected. These PPE items are put on far enough away from the infected person that the risk of transmission is low, and are removed in a similar “safe zone” before exiting the potentially contaminated area. Depending on the primary transmission route this distance may be quite small, for example when concerned only about contact with body fluids, or large, as when transmission occurs primarily by sneezing or coughing. Before examining patients with pneumonia, C. difficile colitis, or MRSA or VRE infections of any body system, medical personnel may don PPE outside an isolation room or ward or in a gowning area. When making rounds on an isolation ward, health care personnel may be required to don and remove PPE many times per day.

If the disease-causing agent is relatively robust, it may remain infectious on surfaces long after its original impact, moving to another host through contact with broken skin or mucus membranes. For example, every parent knows that when a child with a cold shares a toy with an uninfected child, she quite often shares the cold, too. A more chilling observation is that, according to the Centers for Disease Control (CDC), HBV can survive for at least a week in dried blood on environmental surfaces or contaminated needles and instruments. C. difficile, a cause of colitis, forms hardy spores that can survive outside the body and are resistant to heat and chemical disinfectants. Thus any object having potentially been in contact with blood or OPIM, including PPE items and reusable medical instruments and devices, must itself be considered contaminated. Under current best practices, contaminated items must be disposed of or decontaminated before reuse.

Many different medical instruments and devices are used to determine a patient's state of health. While some of these devices lend themselves to complete decontamination between uses, others do not. One approach to preventing contamination by the latter devices is to make them disposable. Unfortunately, there is generally a tradeoff to be made between higher instrument quality and lower instrument cost. Lower quality disposable instruments may lack sufficient sensitivity for a health care professional to make a conclusive diagnosis. Thus, even when disposable stethoscopes are available for their use, healthcare professionals may prefer to use their own high-quality stethoscopes and to mitigate contamination risk with “next-best” methods.

### SUMMARY

Although protective coverings for medical instruments are known in the art, they are difficult or even impossible for the user to remove without risking contamination of the instrument or herself. Conversely, coverings that are easy to remove are often difficult to keep in position during use of the instrument. An improved protective covering provides for an unassisted user to secure it to a medical instrument and then remove it when finished, both with a single gloved hand.

In some embodiments, the invention provides a disposable protective covering for reusable medical instruments and devices that can be easily and quickly applied during donning of personal protective equipment (PPE), and easily and quickly removed during removal of PPE, without risking inadvertent contamination of the instrument or device. In some embodiments, a disposable protective covering includes a fastening mechanism that secures the covering to the instrument during instrument use, ensuring that the covering material provides a barrier to contamination of the instrument by contact, and that is releasable in a single motion by a moderate force applied toward the distal (farthest from the head of the user) end of the covering from a point along the distal portion of the covering.

In some embodiments, a disposable protective covering includes a fastening means on the interior surface of the sheath near the proximal end of the covering. This fastening means secures the covering to the instrument during instrument use, and is releasable in a single motion by a moderate force (about 10 pounds-force or less, or less than about 4.5 Newtons) applied toward the distal end of the covering from a point along the distal portion of the covering. In some embodiments, a disposable protective covering includes a fastening means extending from the proximal end of the covering. In some embodiments this fastening means comprises a pair of ties. The covering is secured to the medical instrument by forming an overhand or half knot with the ties. The half knot secures the covering to the instrument during instrument use, and is releasable in a single motion by a moderate force applied away from the proximal end of the covering from a point along the distal portion of the covering.

In another aspect, the invention provides a system including a dispenser for facilitating storage and application of the protective covering. Protective coverings may, in some embodiments, be supplied singly and dispensed from a collection of single coverings. In some embodiments, single coverings may be ordered, for example, folded like facial tissues, before being loaded into dispensers. In yet other embodiments, protective coverings may be supplied as a single sheet of coverings, attached side to side or “nose to tail” (proximal end of one abutting distal end of the next), with perforations or slits between coverings to facilitate removal of one covering at a time.

In yet another aspect, the invention provides a method of protecting medical instruments from contamination during use and for protecting users from contamination during disposal of a protective covering. In some embodiments, users cover a stethoscope by inserting its distal portion (including the bell) into the proximal opening of the protective covering, securing the cover against inadvertent removal, for example by making a half knot around the yoke of the stethoscope, examine the patient, and then remove the protective covering by pulling it off using a gloved hand, such that neither a potentially contaminated glove nor the used protective covering enters the contamination-free area around the user's head and neck.

### BRIEF DESCRIPTION OF THE DRAWINGS

The present invention may be better understood, and its numerous objects, features, and advantages made apparent to those skilled in the art by referencing the accompanying drawings.

FIG. 1 is a sketch of the torso of a health care provider wearing gown, gloves, and stethoscope, and holding an otoscope.

FIG. 2 depicts an exemplary protective covering.

FIG. 3 depicts removal of a contaminated exemplary protective covering.

FIGS. 4A through 4E depict exemplary fastening mechanisms. FIG. 4F depicts the proximal portion of an exemplary protective covering in a fastened configuration.

FIG. 5 depicts an exemplary protective covering secured to an otoscope.

FIGS. 6A and 6B depict an exemplary protective covering being dispensed from a roll of coverings.

FIG. 7 depicts an exemplary protective covering being dispensed from an exemplary wall dispenser.

FIG. 8 depicts an exemplary protective covering packaged for sterile presentation.

The use of the same reference symbols in different drawings indicates similar or identical items.

### DESCRIPTION OF THE PREFERRED EMBODIMENT(S)

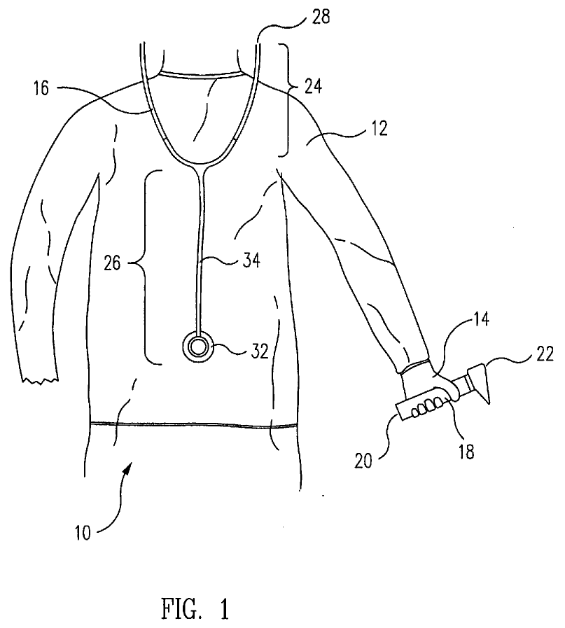

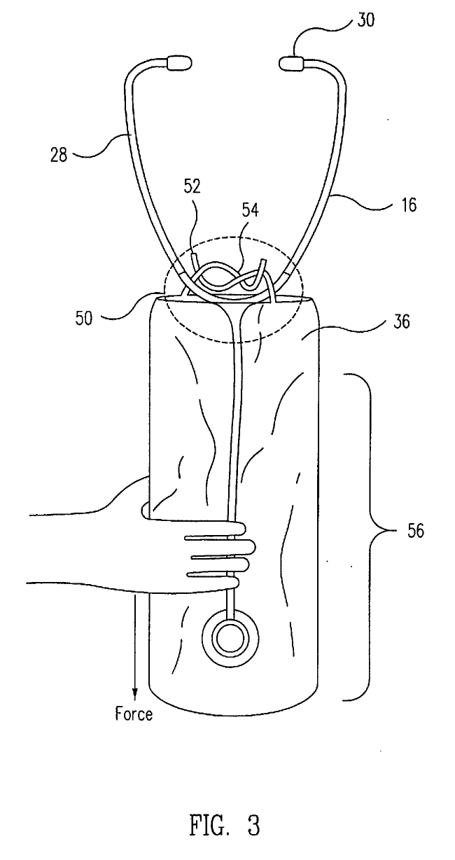

FIG. 1 is a sketch of the torso of a health care provider 10 wearing gown 12, gloves 14, and stethoscope 16, and holding an otoscope 18. (Otoscopes and opthalmoscopes differ in the attachments affixed to the head of the instrument and in their primary targets, but may share an instrument body.) Directions and descriptors are generally given with reference to the user 10, so the proximal end 20 of the otoscope 18 is the end in the user's grasp, while the distal end 22 of the otoscope 18 is the portion in contact with or nearest to the patient. Similarly the proximal portion 24 of the stethoscope 16 is close to the user's head (not shown) while the distal portion 26 hangs along the user's torso when not in use. The proximal portion 24 of the stethoscope 16 includes the ear piece tubes 28 and ear tips 30 (see FIG. 3). The distal portion 26 includes the head, or bell, 32 of the stethoscope 16 and most, or even all, of the listening tube 34 (sometimes called “tubing” or “tube”), and is more likely than the proximal portion 24 to become contaminated.

Personal protective equipment (PPE) such as the gown 12 and gloves 14 of FIG. 1 is available in a variety of sizes, colors, and materials to suit differing requirements of users and contaminant environments. Where there is no accepted standard protective gear, such as for a stethoscope or other medical instrument, health care personnel may attempt to adapt PPE designed for other purposes to the need at hand. These attempts may sometimes include using a disposable glove to cover the distal parts of a stethoscope, holding the glove on with one hand while using the other to manipulate the patient, bed contents, or other objects. The contortions required to protect the stethoscope in such a case may reduce the quality of the examination.

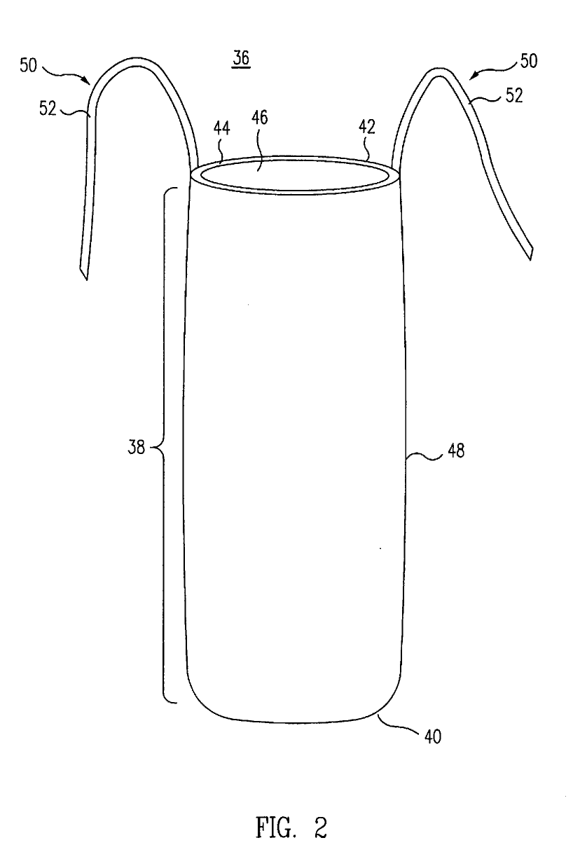

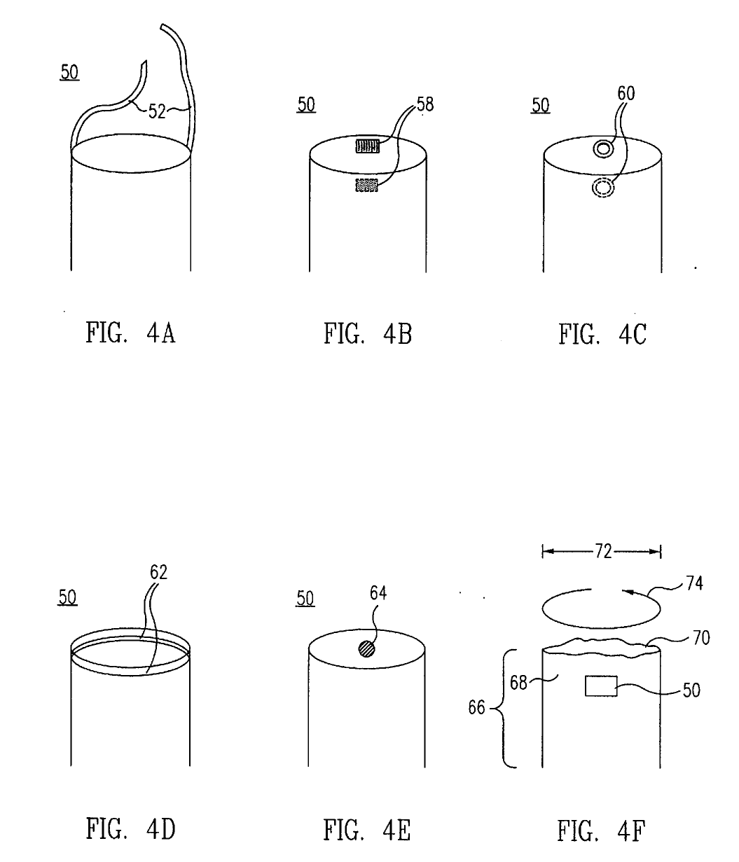

FIG. 2 depicts an exemplary protective covering 36. The body or sheath 38 of the covering is preferably constructed of a barrier material that is impermeable to water and microorganisms, while not degrading signal transmission between the patient and the instrument. For example, a barrier material appropriate for protecting a stethoscope would exhibit good sound transmission qualities but its ability to transmit light is immaterial, while the reverse would be true of an appropriate barrier material for use with an otoscope or an opthalmoscope. Natural latex, synthetic rubbers, vinyl, and nitrile materials, similar to those used for examination and surgical gloves, are a few of the appropriate materials. The sheath 38 extends between the distal 40 and proximal 42 ends of the covering 36. The proximal end 42 of the sheath 38 is open, while the distal end 40 may be open or closed. The sheath 38 has a perimeter 74 (see FIG. 4F) large enough to admit the largest part of the medical instrument for which protection is desired. The barrier material forms a sheath wall 44, that has an interior surface 46 and an exterior surface 48 (the interior and exterior surfaces, respectively, of the sheath 38). At or near the proximal end 42 of the sheath 38, a fastening mechanism 50, depicted here as ties 52, is provided to secure the covering 36 to a medical instrument.

FIG. 3 depicts removal of a contaminated exemplary protective covering 36. The protective covering 36 has been secured to the stethoscope 16 by a fastening mechanism 50, shown here as a pair of ties 52 fastened into a half knot 54. One of the ties 52 passes between the stethoscope 16 and the torso of the user 10 (not shown here) and over the ear piece tube 28, while the other tie 52 passes over the ear piece tube 28 from the outward facing side of the stethoscope 16. The two ties 52 are then tied into a half knot 54, also sometimes called half a square knot, over the yoke, forming a fastened configuration. The half knot 54 could alternatively be formed off-center around one of the ear-pieces or between cross-pieces if the stethoscope has more than one. The half knot 54 is sufficient to keep the protective covering 36 secured to the stethoscope 16 during use under normal conditions. Before entering a non-contaminated area after using the stethoscope 16, the user 10 may remove the protective covering 36 by grasping it at a point along its distal portion 56 and tugging it away from the proximal portion 24 of the stethoscope 16 in a direction roughtly parallel to the longitudinal centerline of the covering 36 (force arrow Force), although accuracy in direction is not required as long as the force is applied in a direction away from the proximal end of the sheath 38. The ties 52 disentangle in response to the applied force, returning to the unfastened configuration, and the covering 36 may be disposed of along with gloves 14, gown 12, and any other disposable PPE donned before entry into the contaminated work area. As is clear from the figure and the description, the distal portion 56 of the protective covering 36, which is also the distal portion of the sheath 38, may make up more than half, or even most, of the length of the covering, but doesn't include the region of the fastening mechanism 50 or the proximal end 42 of the sheath.

FIGS. 4A through 4E depict exemplary fastening mechanisms 50.

FIG. 4A shows a pair of ties 52 similar to those seen in FIGS. 2 and 3. Ties 52 need not be formed integrally with the sheath wall 44, although they may be. Ties 52 need not be made of the same material as the sheath 38. Ties 52 may be twist ties, i.e. plastic-coated wires or other materials that maintain their shape when initially deformed yet are easily deformed into a subsequent desired shape such as an unfastened configuration. It may be convenient to make ties 52 of a non-barrier material, for example cotton or other fabric, to reduce the likelihood of the ties 52 sticking during removal from a medical instrument with a rubber or plastic securing point. Alternatively, it may be advantageous to provide ties of a tacky material for use with medical instruments made largely of smooth-surfaced metal.

While fastening or closing the fastening mechanism allows the protective covering to be secured to the medical instrument, unfastening it releases the sheath from its secured position. Any fastening mechanism 50 that can be unfastened by application of force on the distal portion 56 of the sheath 38 directed away from the proximal portion 24, 20 of the protected instrument 16, 18, that is, without requiring direct contact with the fastening mechanism itself, may be used to secure the covering 36 to the instrument 16, 18. The force required to remove the protective covering is preferably less than that required to dislodge the medical instrument from its desired location, e.g. remove it from the wearer's ears or neck; in most cases the force required is less than about 10 pounds-force or 4.5 Newtons. In some embodiments, the force required to return a fastened mechanism to the unfastened configuration is insufficient to rupture the covering or the fastening mechanism, or to detach the fastening mechanism from the body of the protective covering.

FIG. 4B depicts a hook and loop closure 58, FIG. 4C a snap fastener 60, and FIG. 4E an adhesive closure 64. FIG. 4D depicts a protective covering with a tongue and groove fastener 62, as commonly found in sandwich bags, where on one portion of the inner surface a pair of relatively thicker ridges of the sheath material (or a different material) form a groove between them into which a single relatively thicker ridge from the opposite inner surface may be inserted. The ridges are shaped and positioned so that a moderate pressure is required to fasten, i.e. insert the single ridge into the groove, and to unfasten, i.e. remove the tongue from the groove, the closure. Both the snap fastener 60 of FIG. 4C and the tongue and groove fastener 62 of FIG. 4D are examples of nesting fastening mechanisms. In other words, in these and other nesting fasteners, one of a pair of pieces forming part of the fastening mechanism nests in the other of the pair in the fastened configuration, while they are separated in the unfastened configuration.

When the fastening mechanisms 50 of FIGS. 4B through 4E, and some other similar mechanisms not depicted but known to those skilled in the fastening arts, are transformed from the unfastened configuration to the fastened configuration, the covering 36 is flattened at least along the fastened region, as depicted in FIG. 4F. When flattened, the proximal region 66 of the sheath 38 may be described as having front 68 and rear 70 walls, each with a width 72 approximately half the perimeter 74 of the sheath 38. In this view, opposite regions of the interior surface 46 of the sheath can be said to mutually attach when in the fastened configuration. The hook and loop 58 and tongue and groove 62 fasteners may extend along the entire width 72 or along only a portion of the walls 68, 70. The adhesive 64 may include a single adhesive patch on either the front 68 or rear 70 wall, paired patches on both front 68 and rear 70 walls, multiple adhesive patches not necessarily aligned one with another, a continuous strip along either the front 68 or rear 70 wall, a continuous strip along the entire inner perimeter of the covering, or other convenient adhesive configurations.

Alternatively, in some embodiments perforations or other material weakening means may be provided near the fastening mechanism at its distal side. The force required to dislodge the protective covering from the medical instrument by rupturing the covering at the pre-weakened area may in this case be less than that required to return the fastening mechanism to its unfastened configuration; in other words, it may be easier to tear the potentially contaminated sheath from the instrument than to remove it by unfastening the fastening mechanism. Such an arrangement would still permit the use of fastening mechanisms that can easily revert to their unfastened configurations, but would not require them. Nevertheless, such a weakened area will preferably be near enough the fastening mechanism that the mechanism itself is dislodged from the medical instrument during the removal of the protective covering. The weakened or perforated area need not extend along the entire perimeter of the covering, in which case the fastening mechanism may remain attached to the body of the sheath along a portion of the sheath's proximal end, and be removed along with the rest of the protective covering.

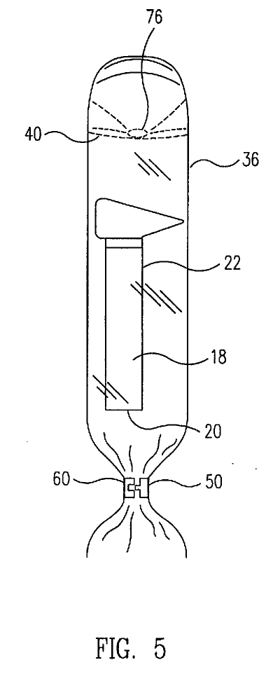

FIG. 5 depicts an exemplary protective covering 36 secured to an otoscope 18. As this figure makes clear, “securing” a covering “to” an instrument need not require “attaching” or “affixing” it; here the distal end 40 of the covering is fastened to itself and the proximal end 42 is closed with a fastener, but nowhere is the covering attached or affixed to the instrument. Before entering the contaminated work area, a health care provider may slip the distal end 22 of an otoscope 18 into a protective covering 36. In the example of FIG. 5, both proximal 42 and distal 40 ends of the covering are open, so that either end of the instrument may be inserted into either end of the covering 36. Once the otoscope 18 is surrounded by the covering 36, the proximal end 42 of the covering 36 is fastened using the fastening mechanism 50, shown here as a snap fastener 60. In some circumstances the distal end 40 of the protective covering 36 need not be closed during use of the otoscope. However, it may often be convenient to secure the distal end 40 of the covering 36 to close it or merely secure it out of the way of the health care provider. The embodiment depicted in FIG. 5 provides a means 76 for closing the distal end 40 of the protective covering 36. The particular example depicted in FIG. 5 shows the closure 76 as an adhesive dot on the outside surface of the protective covering on the opposite side of the instrument from the viewer. Here the excess portion of the protective covering is folded away from the viewer (toward the user's left) and secured out of the way. As was the case for the proximal end fastening mechanism 50, many types of fastening mechanisms may be employed to close the distal end 40. An adhesive fastener 76 is depicted in FIG. 5, but snaps, hook and loop, tongue and groove, many kinds of tape, and other fastening mechanisms may be used.

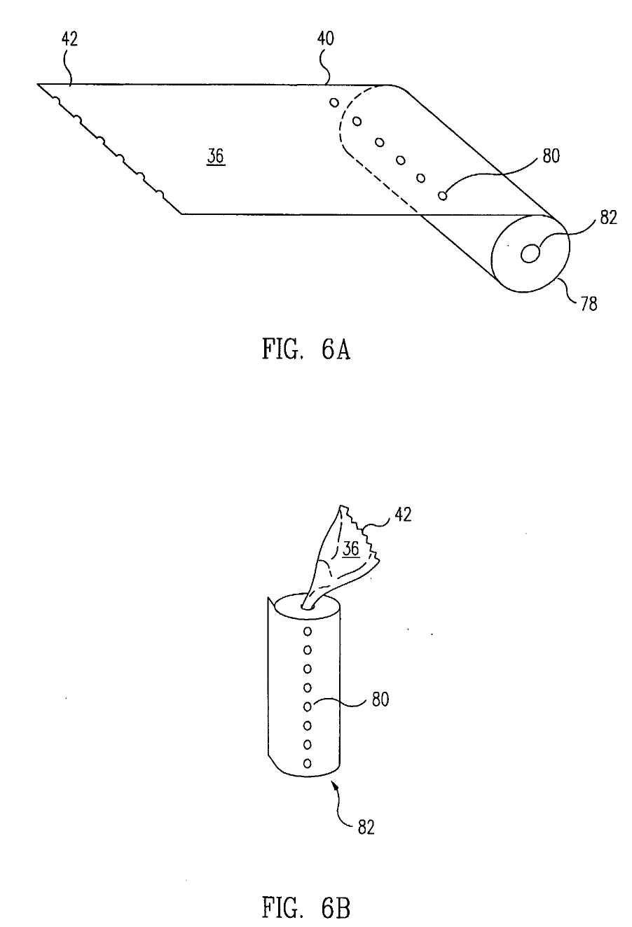

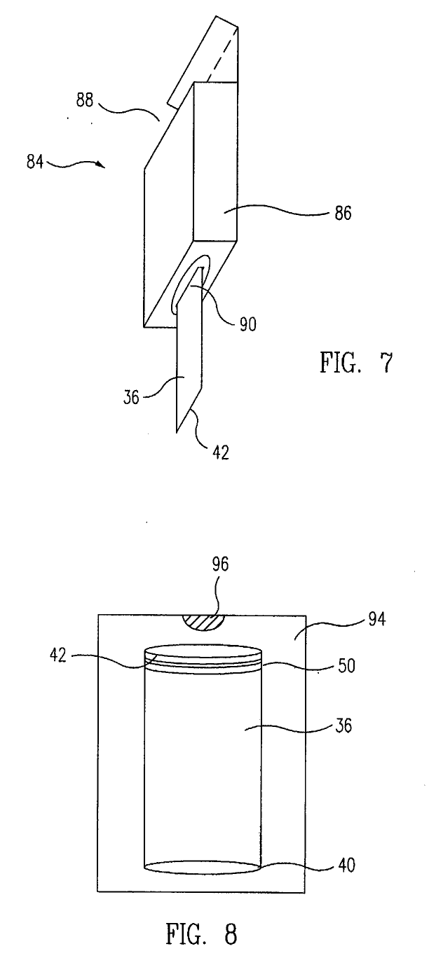

Protective coverings 36 may be dispensed by multiple mechanisms common in medical disposables packaging. (It should be noted that while closures or fastening mechanisms have not been called out in FIGS. 6A, 6B and 7, they are omitted only to reduce clutter and improve clarity of the points illustrated by those figures and not to imply their absence.) FIGS. 6A and 6B depict an exemplary protective covering 36 being dispensed from a roll 78 of coverings. Referring to FIG. 6A, coverings 36 may be manufactured in a continuous sheet with the proximal end 42 of one covering 36 abutting the distal end 40 of the next covering 36 in the sheet. In some cases it may be more convenient to position the coverings side by side in the roll, for example when forming certain types of fastening mechanisms such as, e.g., tongue and groove closures. Coverings 36 may be delimited by perforations or slits 80 formed between coverings 36 to facilitate their separation from one another. The sheet of coverings 36 may be formed into a roll 78 by methods well known in the packaging plastic manufacturing arts. In general, the sheet is wound around a core 82 which may be hollow or solid and which may be retained as in FIG. 6A, or removed as in FIG. 6B. Coverings may then be dispensed from the outside of the roll 78 as in FIG. 6A, or from its center as in FIG. 6B, depending on the requirements of the covering users. Such a roll 78 may be used as the dispenser itself, or as part of a larger dispensing system. Protective coverings need not be rectangular, of course, and may form nested structures in the roll, as when ties from one covering extend along the outside of the body of the next or previous covering.

FIG. 7 depicts an exemplary protective covering 36 being dispensed from an exemplary dispenser 84. Wall dispensers are well known in the medical disposables packaging arts and are commonly used to dispense gloves, bouffant caps, shoe covers, and other disposable items used in medical settings. Such dispensers as are commonly available may be used to dispense protective coverings 36 for medical instruments, with or without adaptation as required. In the example depicted in FIG. 7, a wall dispenser 84 includes a container 86 having a non-restrictive opening 88 and a restrictive opening 90. The restrictive opening 90, shown here as a slit, is sized so as to allow the passage of only a single protective covering 36 at a time. It should be noted that dispenser 84 may function to dispense coverings without necessarily being affixed to a wall or any other structure. Coverings 36 may be loaded into the container 86 through the non-restrictive opening 90. The container 86 may be loaded with individual coverings or coverings in a continuous sheet, for example fan-folded or in a roll 78.

Useful on general rounds and in isolation wards to reduce the risk of spreading infections between patients as well as to staff, protective coverings 36 may also find use in sterile environments such as operating rooms. FIG. 8 depicts an exemplary protective covering 36 packaged for sterile presentation. In the example shown, a protective covering 36 is placed between barrier layers of a sterilizable package 94, with the proximal end 42 closest to the edge 96 of the package to be presented to the sterile user. Sterile packs may be provided directly by the manufacturer or they may be made up by hospital staff and sterilized along with other surgical supplies.

While the invention has been described with reference to various embodiments, it will be understood that these embodiments are illustrative and that the scope of the invention is not limited to them. Many variations, modifications, additions, and improvements are possible. The protective coverings as described herein may be used with many medical instruments and devices when it is desirable to protect those items from environmental contamination; the examples of stethoscopes, otoscopes, and opthalmoscopes are provided to illustrate typical uses with common instruments. In certain situations protective coverings may be applied to medical instruments and/or removed from them by someone other than the user, for example by an assistant. In certain circumstances it may be appropriate to use multiple coverings for a single instrument. While primarily intended as a single use item, in some situations it may be desirable to recondition used coverings for reuse. These and other variations, modifications, additions, and improvements may fall within the scope of the invention as defined in the claims that follow.

What is claimed is:

1. A protective covering for preventing contamination of a medical instrument comprising:
a sheath of barrier material,
the sheath having a proximal end, a distal end, a length therebetween, interior and exterior surfaces, and a perimeter; and
a fastening mechanism to secure the sheath to the proximal end of the medical instrument,
the fastening mechanism having a fastened configuration and an unfastened configuration, and
the unfastened configuration being achievable from the fastened configuration by application of a force directed away from the proximal end of the sheath, said force being applied on the sheath nearer the distal end of the sheath.

2. The protective covering of claim 1, wherein the unfastened configuration is achievable from the fastened configuration without direct contact with the fastening mechanism.

3. The protective covering of claim 1, wherein the fastening mechanism comprises a pair of ties extending from the proximal end of the sheath.

4. The protective covering of claim 1, wherein the fastening mechanism is disposed on the interior surface of the sheath.

5. The protective covering of claim 4, wherein at least a portion of opposite regions of the interior surface of the sheath mutually attach in the fastened configuration.

6. The protective covering of claim 5, wherein the fastening mechanism comprises a nesting fastening mechanism.

7. The protective covering of claim 5, wherein the fastening mechanism comprises an adhesive.

8. The protective covering of claim 1, wherein the distal end of the sheath is closed.

9. The protective covering of claim 1, further comprising a means for at least partially closing the distal end of the sheath.

10. The protective covering of claim 1,
wherein the perimeter of the sheath is greater than the perimeter of a head of a stethoscope, and
wherein the length of the sheath is greater than a distance from a proximalmost point on a listening tube to a distalmost point on the head of the stethoscope.

11. The protective covering of claim 1,
wherein the perimeter of the sheath is greater than the perimeter of a head of an otoscope, and
wherein the length of the sheath is greater than a length of the otoscope.

12. A protective covering for preventing contamination of a medical instrument comprising:
a sheath of barrier material,
the sheath having a proximal end, a distal end, a length therebetween, interior and exterior surfaces, and a perimeter; and
fastening means for removably securing the proximal end of the sheath to the medical instrument,
the fastening means having a fastened configuration and an unfastened configuration, and
the unfastened configuration being achievable from the fastened configuration by application of a force directed away from the proximal end of the sheath, said force being applied on the sheath nearer the distal end of the sheath.

13. The protective covering of claim 12, wherein the unfastened configuration is achievable from the fastened configuration without direct contact with the fastening means.

14. The protective covering of claim 12, wherein the fastening means comprises a nesting fastening mechanism.

15. The protective covering of claim 12, wherein the distal end of the sheath is closed.

16. A system for preventing contamination of a medical device comprising:
the protective covering of claim 1, and
a means for dispensing the protective covering.

17. The system of claim 16, wherein the dispensing means comprises a sterilizable package for sterile presentation.

18. The system of claim 16, wherein the dispensing means comprises a roll,
said roll comprising a plurality of protective coverings.

19. The system of claim 18,
wherein the proximal end of each covering is delimited from the distal end of a subsequent covering by perforations, said perforations allowing separation of each covering from the subsequent covering.

20. The system of claim 16,
wherein the dispensing means comprises a container mountable to a wall,
the container comprising a non-restrictive opening and a restrictive opening,
the container fillable with a plurality of protective coverings through the non-restrictive opening, and
the protective coverings dispensable through the restrictive opening.

21. A method for preventing contamination of a medical instrument, the method comprising:
before potential contamination, positioning a protective covering comprising a sheath of barrier material to surround a contaminable portion of a medical instrument and securing said sheath to the medical instrument; then
subjecting a distal portion of the sheath surrounding the medical instrument to potential or actual contamination; then
removing said sheath from the medical instrument by grasping the potentially or actually contaminated distal portion of the sheath and applying force directed away from a proximal end of the sheath, without requiring contact near the proximal end of the sheath to release the sheath prior to its removal.

22. The method of claim 21, wherein said force is insufficient to irreversibly damage said sheath.

23. The method of claim 21, wherein said force is less than approximately 4.5 Newtons.

24. The method of claim 21, wherein:
securing the sheath to the medical instrument comprises transforming a fastening mechanism of the protective covering from an unfastened configuration to a fastened configuration; and
applying the force resulting in the release of the sheath by returning the fastening mechanism to the unfastened configuration without irreversible damage to the sheath.

25. The method of claim 21, further comprising:
before potential contamination, donning protective gloves; then
after removing the sheath, removing the potentially or actually contaminated gloves.

26. The method of claim 21, wherein said force is applied by a single hand.
